(19) United States
(12) Patent Application Publication
(10) Pub. No.: US 2023/0203462 A1
(43) Pub. Date: Jun. 29, 2023

(54) MODIFIED ENDONUCLEASES AND RELATED METHODS

(71) Applicant: UNIVERSITY OF WASHINGTON, Seattle, WA (US)

(72) Inventors: Zhedfan Yuan, Seattle, WA (US); Shaoyi Jiang, Seattle, WA (US); Yanjiao Han, Seattle, WA (US); Sijin Luozhong, Seattle, WA (US)

(73) Assignee: UNIVERSITY OF WASHINGTON, Seattle, WA (US)

(21) Appl. No.: 17/767,426

(22) PCT Filed: Oct. 9, 2020
(86) PCT No.: PCT/US2020/055095

§ 371 Date: Apr. 7, 2022

Related U.S. Application Data
(60) Provisional application No. 62/913,916, filed on Oct. 11, 2019.

(51) Int. Cl. C12N 9/22; C12N 15/52; C12N 15/113

(52) U.S. Cl. CPC C12N 9/22; C12N 15/52; C12N 15/113

(57) ABSTRACT

Provided are compositions and methods for the production of modified endonucleases such as CRISPR/Cas9 system with reduced off-target activity. Methods of editing of polynucleotides using the modified endonucleases in vitro and in vivo are also disclosed. In one aspect, the disclosure provides a modified endonuclease, comprising an endonuclease and one or more mixed charge moieties covalently linked to the endonuclease, wherein each mixed charge moiety comprises about 10 to about 400 positively charged moieties and about 10 to about 400 negatively charged moieties, and wherein the ratio of the number of positively charged moieties to the number of negatively charged moieties is from about 1 :0.5 to about 1 :2.

### CROSS-REFERENCE TO RELATED APPLICATION

This application claims the benefit of U.S. Provisional Application No. 62/913,916 filed on Oct. 11, 2019, the disclosure of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 72968_SEQ_final-2020-10-7.txt. The text file size is 27.2 KB and was created on Oct. 7, 2020 and is being submitted via EFS-Web with the filing of the specification.

### STATEMENT OF GOVERNMENT LICENSE RIGHTS

This invention was made with government support under Grant No. R21 GM128004, awarded by the National Institutes of Health. The government has certain rights in the invention.

### BACKGROUND

CRISPR/Cas system is a widely used tool for genome editing in various organisms and cell types. Unfortunately, it can also cause unwanted mutations at off-target sites that resemble the on-target sequence. The off-target mutations are caused by the nonspecific recognition of DNA sequence by CRISPR/Cas9 RNPs. It has been demonstrated that besides the optimal PAM sequence 5′-NGG-3′, Cas9 can also cleave sites with a 5′-NAG-3′ or 5′-NGA-3′ PAM although less efficiently. In addition, a 20 nt single guide RNA (sgRNA) can recognize DNA sequences that harbor as many as 3-5 base pair mismatches with the sgRNA, suggesting there are up to thousands of possible binding sites for a given nuclease in the human genome. Furthermore, CRISPR/Cas9 can induce off-target cleavages with DNA sequences containing a few extra bases (‘DNA bulge’) or a few missing bases (‘RNA bulge’) compared to the RNA guide strand. Off-target DNA cleavages can give rise to mutations at unintended genomic loci and to gross chromosomal rearrangements such as deletions, inversions, and translocations. These mutations at unwanted sites might disable a tumor-suppressor gene or activate a cancer-causing gene. Translocations have been known to be a possible reason for chronic myeloid leukemia.

Preventing, avoiding, or at least reducing these off-target effects is crucial for the success of any downstream genome editing applications. Various strategies have been developed to reduce genome-wide off-target mutations of the commonly used Cas9 nuclease, including truncated sgRNAs bearing shortened regions of target complementarity, Cas9 mutants, paired Cas9 nickases, and dimeric fusions of catalytically inactive Cas9 to a non-specific FokI nuclease. However, these approaches are only partially effective and/or possess the potential to create more off-target sites. Furthermore, they may also require the expression of multiple sgRNAs and/or fusion of additional functional domains to Cas9, which can reduce the targeting range and create challenges for delivery using viral vectors which have a limited payload size of nucleic acids.

Thus, a need remains for a simple robust strategy that can reduce the off-target effects of the CRISPR/Cas9 system and CRISPR/Cas9 systems with reduced off-target effects.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a modified endonuclease, comprising an endonuclease and one or more mixed charge moieties covalently linked to the endonuclease, wherein each mixed charge moiety comprises about 10 to about 400 positively charged moieties and about 10 to about 400 negatively charged moieties, and wherein the ratio of the number of positively charged moieties to the number of negatively charged moieties is from about 1:0.5 to about 1:2. In some embodiments, the mixed charge moiety is substantially electronically neutral at pH of about 7.4.

In some embodiments, the endonuclease is a nucleic acid-guided nuclease system protein. In some embodiments, the endonuclease is a CRISPR-associated (Cas) protein, such as Cas9, Cas12, Cas13, Cas14, or a mutant or a variant thereof. In some embodiments, the endonuclease is Cas9 or a mutant or a variant thereof.

In some embodiments, the endonuclease is active in a CRISPR/Cas system, wherein the CRISPR/Cas system displays reduced off-target editing activity and maintained on-target editing activity relative to a wild-type CRISPR/Cas system. In some embodiments, the off-target editing activity is reduced by at least 20%, at least 25%, atleast 30%, at least 35%, at least 40%, at least 45%, at least 50%, at least 55%, at least 60%, at least 65%, at least 70%, at least 75%, or at least 80% compared to an unmodified endonuclease.

In some embodiments, the mixed charge moiety is covalently linked to a side chain of an amino acid of the endonuclease, to the N-terminal amino group of the endonuclease, and/or to the C-terminal carboxylic group of the endonuclease.

In some embodiments, the mixed charge moiety is a peptide with a molecular weight of about 2 kDa to about 130 kDa.

In some embodiments, the modified endonuclease is a fusion protein, wherein the mixed charge moiety is a mixed charge domain consisting of:
a) a plurality of negatively charged amino acids;
b) a plurality of positively charged amino acids; and
c) optionally a plurality of additional amino acids independently selected from the group consisting of proline, serine, threonine, asparagine, glutamine, glycine, and derivatives thereof; and
wherein the ratio of the number of positively charged amino acids to the number of negatively charged amino acids is from about 1:0.5 to about 1:2.

In some embodiments, the mixed charge domain comprises a random sequence. In some embodiments, the mixed charge domain comprises a sequence (X1-X2-X3)n, wherein X1 is a positively charged amino acid, X2 is a negatively charged amino acid, and X3 is absent or is an additional amino acid independently selected from the group consisting of proline, serine, threonine, asparagine, glutamine, glycine, and derivatives thereof, wherein n is an integer from about 5 to about 50.

In some embodiments, the plurality of negatively charged amino acids is independently selected from the group consisting of aspartic acid, glutamic acid, and derivatives thereof. In some embodiments, the plurality of positively charged amino acids is independently selected from the group consisting of lysine, histidine, arginine, and derivatives thereof. In some embodiments, the mixed charge domain does not comprise a plurality of additional amino acids. In some embodiments, the plurality of positively charged amino acids are lysines and a plurality of negatively charged amino acids are glutamic acids.

In some embodiments, the mixed charge domain comprises a plurality of lysines and a plurality of negatively charged amino acids selected from the group consisting of glutamic acid and aspartic acid. In some embodiments, the mixed charge domain comprises a plurality of histidines and a plurality of negatively charged amino acids selected from the group consisting of glutamic acid and aspartic acid. In some embodiments, the plurality of additional amino acids is selected from the group consisting of proline, serine, and glycine. In some embodiments, the plurality of additional amino acids is a plurality of prolines. In some embodiments, the mixed charge domain comprises a plurality of lysines, a plurality of glutamic acids, and a plurality of prolines.

In some embodiments, the mixed charge moiety is a synthetic polymer with a molecular weight of about 2 kDa to about 80 kDa. In some embodiments, the polymer selected from the group consisting of poly(carboxybetaine) (PCB), poly(sulfobetaine) (PSB), poly(2-methacryloyloxyethyl phosphorylcholine) (PMPC), and poly(trimethylamine oxide) (TMAO) polymers. In some embodiments, the polymer is a poly(carboxybetaine) (PCB).

In another aspect, the disclosure provides a nucleic acid comprising a sequence encoding the modified endonuclease disclosed herein.

In another aspect, the disclosure provides an expression vector comprising the nucleic acid of the disclosure and a promoter operably linked thereto.

In another aspect, the disclosure provides a cell comprising the nucleic acid or the expression vector of the disclosure. In some embodiments, the cell is a prokaryotic cell or eukaryotic cell. In some embodiments, the cell is a mammalian cell. In some embodiments, the cell is in a cell culture. In some embodiments, the cell is in an organism.

In another aspect, the disclosure provides a method for editing a polynucleotide in a cell or in a subject, the method comprising introducing into the cell or the subject at least one modified endonuclease, a nucleic acid, or an expression vector of the disclosure.

In some embodiments, the polynucleotide is DNA or RNA. In some embodiments, the nucleic acid is an mRNA encoding the modified endonuclease.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a schematic overview of the GFP disruption assay and the target site used in the GFP gene.

FIG. 1B shows efficiency of GFP disruption in HEK293-GFP cells mediated by native Cas9 and exemplary modified Cas9 (pCB-Cas9 conjugates).

FIG. 1C demonstrates off-target editing efficiency of native Cas9 and pCB-Cas9 conjugates with mismatched sgRNA harboring one, two, or three nucleotide mutations in GFP disruption assay.

FIG. 2 is a schematic of mechanism of pCB conjugation in reducing the off-target efficiency of the CRISPR/Cas9 system. The Cas9/sgRNA complex possesses more energy than what is needed for optimal recognition of its target DNA site, leading to the cleavage of mismatched off-target sites. pCB polymer conjugation eliminates the non-specific binding between Cas9/sgRNA complex and double-strand DNA, thereby decreasing the binding energy. The remained energy is strong enough for on-target binding, but not enough for mismatched binding.

FIGS. 3A-3C shows the On-target and off-target sequences of Cas9:sgRNAs when targeting VEGF (3A), EMX (3B) and CLTA (3C) loci.

FIGS. 3D-3F demonstrate on-target and off-target DNA editing efficiencies resulting from native Cas9 and pCB-Cas9 when targeting VEGF (top), EMX (middle) and CLTA (bottom) in three different cell lines HEK293 (3D), U2OS (3E) and K562 (3F).

FIG. 4 shows the synthetic route used for the preparation of exemplary pCB-Cas9 conjugates.

FIG. 5 is a size-exclusion chromatogram of native Ca, and exemplary pCB₁₀-Cas9, pCB₂₀-Cas9, pCB₅₀-Cas9 conjugates.

FIGS. 6A and 6B demonstrate exemplary optimal sgRNA to protein ratio for native Cas9 (6A) and pCB-Cas9 (6B). All experiments were performed in a 96-well plate using a volume of 110 ul.

FIGS. 7A and 7B show effect of CRISPRMAX dose on the delivery efficiency and cellular toxicity of Cas9/sgRNA (7A) and pCB-Cas9/sgRNA (7B).

FIGS. 8A-8C show construction of expression plasmids encoding exemplary modified Cas9 (Cas9-(EK)ₙ).

FIG. 9 is a gel electrophoresis of in vitro transcribed Cas9 and Cas9-EK mRNA pre- and post-polyadenylation.

FIG. 10 is a graph of gene editing efficacy of Cas9 using commercialized Cas9 mRNA and lab prepared Cas9 mRNA.

FIGS. 11A-11C are gel electrophoresis of on-target DNA editing resulting from native Cas9, Cas9-(EK)₁₀, (EK)₁₀-Cas9-(EK)₁₀, and Cas9-(EK)₃₀ for target sites GFP (11A), VEGF (11B), and EMX (11C) in HEK293-GFP cells.

FIG. 11D is a graph of quantified data shown in FIGS. 11A-C.

### DETAILED DESCRIPTION

The clustered, regularly interspaced, short palindromic repeats (CRISPR)-associated protein (Cas) system is a powerful genome-editing tool that is widely used in many different applications. However, the high-frequency mutations induced by RNA-guided Cas proteins at sites other than the intended on-target sites is a major concern that impedes therapeutic and clinical applications. Without wishing to be bound by theory, because most off-target events result from the non-specific mismatch between single guide RNA (sgRNA) and target DNA, the inventors hypothesized that minimizing the non-specific RNA-DNA interaction can be an effective solution to this issue.

The inventors demonstrated that by modifying an endonuclease, such as Cas9, with a mixed charged moiety, this mismatch issue can be minimized (for example, by conjugating Cas9 with a zwitterionic polymer or adding a mixed charge peptide domain to the N and/or C terminus of the endonuclease). The CRISPR/Cas ribonucleoproteins (RNPs) of the disclosure show reduced off-target DNA editing but similar levels of on-target gene editing activity. This approach provides a simple and effective way to streamline the development of genome editing with the potential to accelerate a wide array of biotechnological and therapeutic applications of CRISPR/Cas technology.

Thus, in one aspect, the disclosure provides a modified endonuclease, comprising an endonuclease and one or more mixed charge moieties covalently linked to the endonuclease, wherein each mixed charge moiety comprises about 10 to about 400 positively charged moieties or groups and about 10 to about 400 negatively charged moieties or groups, and wherein the ratio of the number of positively charged moieties or groups to the number of negatively charged moieties or groups is from about 1:0.5 to about 1:2.

In some embodiments, the one or more mixed charge moieties comprises about 20 to about 300, about 30 to about 200, about 30 to about 150, or about 30 to about 100 positively charged moieties or groups. In some embodiments, the one or more mixedcharge moieties comprises about 20 to about 300, about 30 to about 200, about 30 toabout 150, or about 30 to about 100 negatively charged moieties or groups.

As used herein, the term “mixed charge moiety” refers to a moiety having substantially equal numbers of positively charged groups and negatively charged groups to provide a moiety that is substantially electronically neutral at a physiologically relevant pH. In some embodiments, the mixed charge moiety is substantially electronically neutral at pH of about 7.4. Mixed charge moieties include zwitterionic moieties.

As used herein, the term “substantially electronically neutral” refers to moieties having a net charge of substantially zero. In some embodiments, the ratio of the number of positively charged moieties or groups to the number of the negatively charged moieties or groups is from about 1:1.1 to about 1:0.5. In some embodiments, the ratio of the number of positively charged moieties or groups to the number of the negatively charged moieties or groups is from about 1:1.1 to about 1:0.7. In some embodiments, the ratio of the number of positively charged moieties or groups to the number of the negatively charged moieties or groups is from about 1:1.1 to about 1:0.9.

The modified endonucleases of the disclosure are active in a CRISPR/Cas system, such as a Class 2 Clustered Regularly Interspaced Short Palindromic Repeat (CRISPR) systems adapted for polynucleotide editing, such as genome engineering. Such engineered CRISPR systems typically contain two components: a guide RNA (gRNA or sgRNA) and a CRISPR-associated endonuclease (Cas protein). The gRNA is a short synthetic RNA composed of a scaffold sequence necessary for Cas-binding and a user-defined nucleotide spacer that defines the polynucleotide (e.g., genomic) target to be modified. Thus, the target of the Cas protein can be changed by simply changing the target sequence present in the gRNA.

The modified endonucleases of the disclosure have certain advantageous properties compared to unmodified (e.g., wild type) endonucleases. Specifically, the modified endonucleases disclosed herein can substantially maintain their on-target activity while having a reduced off-target activity compared to an unmodified endonuclease when used in a CRISPR/Cas system. In some embodiments, the modified endonuclease includes an endonuclease such as a nucleic acid-guided nuclease system protein, e.g., an RNA-guided nuclease. Suitable endonucleases include Cas9, Cas12, Cas13, Cas14, and their mutants and variants thereof. As used herein, a “Cas mutant” or “Cas variant” refers to a protein or polypeptide derivative of the wild type Cas protein which retains substantially one or more of the nuclease activity, RNA binding activity, or DNA targeting activity of the wild type Cas protein. In some embodiments, the protein or polypeptide can comprise, consist of, or consist essentially of a fragment of the protein encoded by SEQ ID NO: 1. In some embodiments, the mutant or variant is at least 50% (e.g., any number between 50% and 100%, inclusive, including but not limited to at least 55%, at least 60%, at least 65%, at least 70%, at least 75%, at least 80%, at least 85%, at least 90%, at least 95%, at least 98%, and at least 99%) identical to the protein encoded by SEQ ID NO: 1. In some embodiments, the endonuclease is Cas9 or a mutant or a variant thereof.

The modified endonucleases of the disclosure, when used in a CRISPR/Cas system, display reduced off-target editing activity and maintained on-target editing activity relative to a wild-type CRISPR/Cas system (i.e., a CRISPR/Cas system comprising the corresponding unmodified endonuclease). In some embodiments, off-target editing comprises editing at undesired genomic locations and/or undesired gene targets. In some embodiments, off-target editing comprises unintended genomic modifications at undesired genomic locations and/or undesired gene targets. In some embodiments, off-target editing comprises nonspecific and/or unintended genetic modifications. In some embodiments, off-target editing comprises unintended/undesired genetic modifications, for example point mutations, deletions, insertions, inversions, and/or translocations. In some embodiments, off-target editing comprises insertions and/or deletions (indels). In some embodiments, the off-target editing activity is reduced by at least 20%, at least 25%, at least 30%, at least 35%, at least 40%, at least 45%, atleast 50%, at least 55%, at least 60%, at least 65%, at least 70%, at least 75%, or at least 80% compared to an unmodified endonuclease. In some embodiments, the off-targetediting activity is reduced by at least 20%, at least 25%, at least 30%, at least 35%, atleast 40%, at least 45%, at least 50%, at least 55%, at least 60%, at least 65%, at least 70%, at least 75%, or at least 80%, compared to an unmodified endonuclease, when the target comprises one, two, three, or more mismatches.

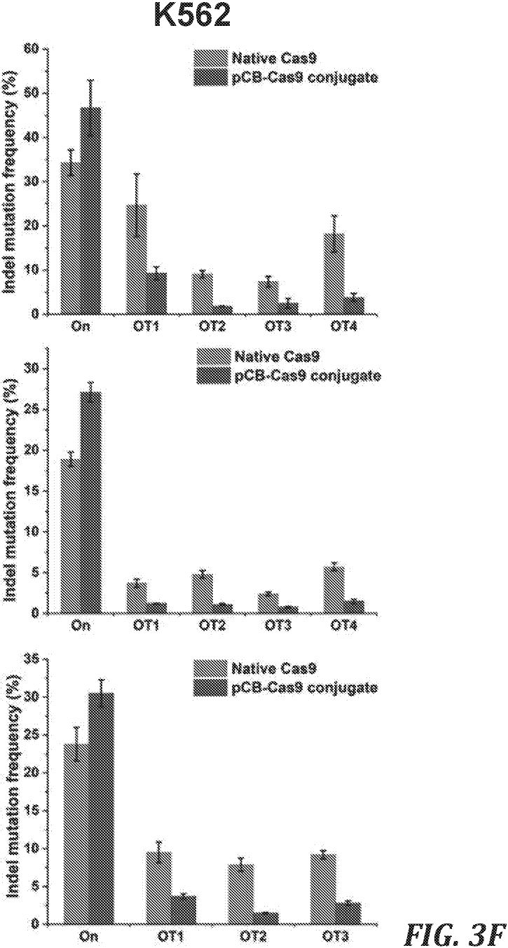

In the modified endonucleases disclosed herein, the one or more mixed charge moieties can be attached to an unmodified endonuclease in any suitable manner. In some embodiments, the modified endonuclease comprises a mixed charge moiety attached to a side chain of one or more amino acids of the endonuclease, e.g., to an amino group of a lysine. Methods of covalently attaching moieties, such as mixed charge moieties, are known in the art. For example, one such exemplary method is shown in FIG. 4. In some embodiments, the modified endonuclease can comprise a short linker, such as an optionally substituted alkylene or an optionally substituted heteroalkylene comprising 2-10 carbon atoms, linking the endonuclease and the mixed charge moiety.

In some embodiments, the mixed charge moiety can be attached to the N terminus or the C terminus of the endonuclease. In some embodiments, the endonuclease can comprise two mixed charge moieties attached to the N terminus and the C terminus of the endonuclease.

Examples of mixed charge moieties suitable for the modification of endonucleases as disclosed herein include synthetic polymers and peptides. In some embodiments, the mixed charge moiety is a synthetic copolymer, e.g., a random copolymer, comprising repeating units with positively charged groups and repeating units with negatively charged groups. In some embodiments, the mixed charge moiety is a zwitterionic synthetic polymer comprising zwitterionic repeating units, i.e., wherein both positive groups and negative groups present in the same repeating unit. In some embodiments, the mixed charge moiety is a peptide comprising substantially equal number of positively charged amino acids and negatively charged amino acids.

In some embodiments, the mixed charge moiety is a random copolymer that does not have extensive regions along the polymer backbone that are positively charged or negatively charged (i.e., the positively and negatively charged constitutional units are relatively uniformly distributed along the polymer backbone).

In some embodiments, the mixed charge moiety is a zwitterionic polymer. Nonlimiting examples of suitable zwitterionic polymers include poly(carboxybetaine) (PCB), poly(sulfobetaine) (PSB), poly(2-methacryloyloxyethyl phosphorylcholine) (PMPC), and poly(trimethylamine oxide) (TMAO). In some embodiments, the mixed charge moiety is a poly(carboxybetaine) (PCB).

In some embodiments, the mixed charge moiety is a synthetic polymer with a molecular weight of about 2 kDa to about 80 kDa. In some embodiments, the mixed charge moiety is a synthetic polymer with a molecular weight of about 5 kDa to about 50 kDa. In some embodiments, the mixed charge moiety is a synthetic polymer with a molecular weight of about 5 kDa to about 40 kDa. In some embodiments, the mixed charge moiety is a synthetic polymer with a molecular weight of about 5 kDa to about 30 kDa.

In some embodiments, the mixed charge moiety is a peptide. In some embodiments, the mixed charge moiety is a peptide with a molecular weight of about 2 kDa to about 130 kDa. In some embodiments, the mixed charge moiety is a peptide with a molecular weight of about 2 kDa to about 80 kDa. In some embodiments, the mixed charge moiety is a peptide with a molecular weight of about 5 kDa to about 70 kDa. In some embodiments, the mixed charge moiety is a peptide with a molecular weight of about 10 kDa to about 60 kDa. In some embodiments, the mixed charge moiety is a peptide with a molecular weight of about 10 kDa to about 50 kDa.

The modified endonucleases of the disclosure include fusion proteins. As used herein, a “fusion protein” is a protein consisting of at least two domains that are encoded by separate genes that have been joined so that they are transcribed and translated as a single unit, producing a single polypeptide.

In some embodiments, the modified endonuclease is a fusion protein, wherein the mixed charge moiety is a mixed charge domain consisting of:
a) a plurality of negatively charged amino acids;
b) a plurality of positively charged amino acids; and
c) optionally a plurality of additional amino acids independently selected from the group consisting of proline, serine, threonine, asparagine, glutamine, glycine, and derivatives thereof; and
wherein the ratio of the number of negatively charged amino acids to the number of positively charged amino acids is from about 1:0.5 to about 1:2.

In some embodiments, the mixed charge domain does not comprise a plurality of additional amino acids. In some embodiments, the mixed charge domain consists essentially of a plurality of negatively charged amino acids and a plurality of positively charged amino acids.

In some embodiments, the mixed charge domain comprises a random sequence. In some embodiments, the mixed charge domain comprises a repeat of a sequence comprising one or more positively charged amino acids, one or more negatively charged amino acids, and one or more additional amino acids.

In some embodiments, the mixed charge domain comprises a sequence (X1-X2-X3)n, wherein X1 is a positively charged amino acid, X2 is a negatively charged amino acid, and X3 is an additional amino acid independently selected from the group consisting of proline, serine, threonine, asparagine, glutamine, glycine, and derivatives thereof, wherein n is an integer from about 5 to about 50. In some embodiments, the mixed charge domain comprises a sequence (E-K-X)n, wherein E is a lysine, K is a glutamic acid, and X can be absent or is an additional amino acid independently selected from the group consisting of proline, serine, threonine, asparagine, glutamine, glycine, and derivatives thereof, wherein n is an integer from about 5 to about 50. In some embodiments, the mixed charge domain comprises a sequence (E-K-P)n, wherein E is a lysine, K is a glutamic acid, P is proline, and n is an integer from about 5 to about 50. In some embodiments, the mixed charge domain comprises a sequence (E-K)n, wherein E is a lysine, K is a glutamic acid, and n is an integer from about 5 to about 50.

The mixed charge domain typically comprises about 6 or more amino acids. In some embodiments, the mixed charge domain comprises from about 6 to about 1000 amino acids, from about 20 to about 1000 amino acids, from about 30 to about 1000 amino acids, from about 50 to about 1000 amino acids, from about 80 to about 1000 amino acids, from about 80 to about 600 amino acids, or from about 50 to about 500 amino acids.

The mixed charge domain of the fusion proteins disclosed herein comprise negatively charged amino acids and positively charged amino acids in substantially equal numbers. In some embodiments, the ratio of the number of negatively charged amino acids to the number of positively charged amino acids is from about 1:0.5 to about 1:2, from about 1:07 to about 1:1.4, from about 1:0.8 to about 1:1.25, or from about 1:0.9 to about 1:1.1. Thus, the mixed charge domain is substantially electronically neutral. In some embodiments, the mixed charge domain is substantially electronically neutral at pH of about 7.4.

In some embodiments, the mixed charge domain comprises a plurality of lysines and a plurality of negatively charged amino acids selected from the group consisting of glutamic acid and aspartic acid. In some embodiments, the mixed charge domain comprises a plurality of histidines and a plurality of negatively charged amino acids selected from the group consisting of glutamic acid and aspartic acid.

In some embodiments, the plurality of additional amino acids in the mixed charge domain is selected from the group consisting of serine, asparagine, glycine, and proline. In some embodiments, the plurality of additional amino acids is selected from the group consisting of serine, glycine, and proline. In some embodiments, the plurality of additional amino acids is selected from the group consisting of serine and glycine. The mixed charge domains of the modified endonucleases can comprise only one type of additional amino acid (e.g., proline), two different additional amino acids (e.g., proline and glycine), three different additional amino acids (e.g, serine, glycine, and proline). In some embodiments, the mixed charge domains comprise one additional amino acid. In some embodiments, the mixed charge domains comprise two additional amino acids.

In some embodiments, the plurality of additional amino acids is a plurality of prolines. In some embodiments, the plurality of additional amino acids is a plurality of glycines. In some embodiments, the plurality of additional amino acids is a plurality of serines.

In some embodiments, the mixed charge domain comprises a plurality of lysines, a plurality of glutamic acids, and a plurality of additional amino acids selected from the group consisting of serine, glycine, and proline.

In some embodiments, the mixed charge domain comprises a plurality of lysines, a plurality of glutamic acids, and a plurality of additional amino acids selected from the group consisting of glycine and proline. In some embodiments, the plurality of positively charged amino acids are lysines (K) and a plurality of negatively charged amino acids are glutamic acids (E).

In some embodiments, the mixed charge domain consists essentially of a plurality of negatively charged amino acids; a plurality of positively charged amino acids; and a plurality of additional amino acids independently selected from the group consisting of proline, serine, threonine, asparagine, glutamine, glycine, and derivatives thereof, and optionally an affinity tag, such a histidine tag which can be used for affinity purification of the fusion protein. In some embodiments, the charged domain consists essentially of a plurality of glutamic acids; a plurality of lysines; and a plurality of additional amino acids independently selected from the group consisting of proline and glycine, and optionally an affinity tag, such a histidine tag, which can be used for affinity purification of the fusion protein.

In some embodiments, the fusion protein of the disclosure can comprise an optional domain, such as an affinity tag (e.g., a histidine tag), which can be used for affinity purification of the fusion protein.

The amino acids in the charged domain can be arranged in any manner or sequence, such as in a manner described above. In some embodiments, the charged domain is a random coil polypeptide.

The fusion proteins disclosed herein can be prepared in any suitable manner, for example, using molecular cloning techniques.

Accordingly, in an aspect, when the modified endonuclease is a fusion protein, the disclosure provides a nucleic acid comprising a sequence the modified endonuclease of the disclosure. In one embodiment, the disclosure provides isolated nucleic acids encoding the fusion protein, i.e., a modified endonuclease. The isolated nucleic acid sequence can comprise RNA or DNA. As used herein, “isolated nucleic acids” are nucleic acids that have been removed from their normal surrounding nucleic acid sequences in the genome or in cDNA sequences. Such isolated nucleic acid sequences can further comprise additional sequences useful for promoting expression and/or purification of the encoded polypeptide as previously mentioned. In some embodiments, the nucleic acid can comprise, consist of, or consist essentially of a fragment of the protein encoded by SEQ ID NOS: 2-4. In some embodiments, the nucleic acid is an mRNA encoding the modified endonuclease. In some embodiments, the nucleic acid is a DNA of the sequence SEQ ID NOS: 2-4.

The nucleic acid encoding a fusion protein, i.e., a modified endonuclease of the disclosure, can be incorporated into a suitable expression vector. Accordingly, the disclosure provides an expression vector comprising the nucleic acid encoding a modified endonuclease of the disclosure and a promoter operably linked thereto. An expression vector or an expression construct is a nucleic acid, such as a DNA molecule, that carries a specific gene into a host cell and uses the cell’s protein synthesis machinery to produce the protein encoded by the gene. An expression vector also contains elements essential for gene expression, such as a promoter region operatively linked to the gene, which allows efficient transcription of the gene. The expression of the protein can be controlled, and the protein is only produced in significant quantity when necessary, by using an inducer. E. coli is commonly used as the host for protein production, but other cell types can also be used, such as yeast, insect cells, and mammalian cells.

In another aspect, provided herein is a cell comprising the nucleic acid or a vector encoding a modified endonuclease of the disclosure. The cell can be a prokaryotic cell or eukaryotic cell. In some embodiments, the cell is a mammalian cell. In some embodiments, the cell is in a cell culture. In some embodiments, the cell is in an organism.

In some embodiments, a fusion protein of the disclosure (i.e., a modified endonuclease) can be expressed in vitro or in vivo.

In some embodiments, a fusion protein disclosed herein can be synthesized using any suitable expression system, such as the Escherichia coli expression system, Bacillus subtilis expression system, or any other prokaryotic expression system.

In some embodiments, a fusion protein disclosed herein can be synthesized using the Pichia pastoris expression system. In some embodiments, a fusion protein disclosed herein can be synthesized using the Human Embryonic Kidney 293 expression system. In some embodiments, a fusion protein disclosed herein can be synthesized using the Chinese Hamster Ovary expression system. In some embodiments, a fusion protein disclosed herein can be synthesized using a prokaryotic or eukaryotic cell free expression system.

Recovery and purification of the fusion proteins disclosed herein can be achieved by any suitable method or a combination of such methods. In some embodiments, protein precipitation techniques can be used. In some embodiments, purification can include size exclusion chromatography. In some embodiments, purification can include ion exchange chromatography. In some embodiments, purification can include use of desalting columns. In some embodiments, purification can include affinity chromatography.

In another aspect, provided herein is a method for editing a polynucleotide in a cell or in a subject, the method comprising introducing into the cell or the subject at least one modified endonuclease of the disclosure, a nucleic acid of the disclosure, or an expression vector disclosed herein. Polynucleotides suitable for editing by the modified endonucleases disclosed herein include DNA and RNA. In some embodiments, the polynucleotide is genomic DNA, e.g., human genomic DNA. In some embodiments, the polynucleotide is mitochondrial DNA. The modified endonucleases and nucleic acids and vectors encoding thereof can be introduced into a cell or a subject using any suitable methods known in the art.

The modified endonucleases of the disclosure have a wide range of uses, including genetic engineering, therapeutic gene editing in vivo and ex-vivo, and diagnostic applications.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F.M., et al. (eds.), Current Protocols in Molecular Biology, John Wiley & Sons, New York (2010); and Coligan, J.E., et al. (eds.), Current Protocols in Immunology, John Wiley & Sons, New York (2010) for definitions and terms of art. Additionally, definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). In case of conflict, the terms in the specification will control.

The use of the term “or” in the claims is used to mean “and/or” unless explicitly indicated to refer to alternatives only or the alternatives are mutually exclusive, although the disclosure supports a definition that refers to only alternatives and “and/or.” The words “a” and “an,” when used in conjunction with the word “comprising” in the claims or specification, denote one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. The word “about” indicates a number within range of minor variation above or below the stated reference number. For example, in some embodiments “about” can refer to a number within a range of 10%, 9%, 8%, 7% ,6%, 5%, 4%, 3%, 2%, or 1% above or below the indicated reference number.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these components etc. may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

The following examples are provided to illustrate certain particular features and/or embodiments of the disclosure. The examples should not be construed to limit the disclosure to the particular features or embodiments described.

### EXAMPLES

### Example 1 Preparation of Exemplary Modified Endonucleases by Conjugation With a Mixed Charge Polymer

In this example, Cas9 nuclease from Streptococcus pyogenes was modified by conjugation with a hydrophilic, zwitterionic synthetic polymer.

### 1.1 Synthesis of N-Hydroxysuccinimidepoly(Carboxybetaine Acrylamide) (NHS-PCBAA)

pCB-NHS was synthesized as previously described. Briefly, 3-acrylamido-N-(2-(tert-butoxy)-2-oxoethyl)-N,N-dimethylpropan-1-aminium (CBAAM-tBu) was synthesized. Then, a typical reversible addition fragmentation chain transfer (RAFT) polymerization reaction was performed to yield the 10 kDa SH-pCB polymer. The final colorless NHS-activated polymer was formed by reaction with AMAS at 1:10 molar ratio in DI water (pH 6) for 30 min, followed by removal of unreacted AMAS via Amicon spin dialysis tubes and freeze-drying for 48 h.

### 1.2 Preparation and Characterization of pCB-Cas9 Conjugates

Conjugate of pCB-Cas9 was synthesized by reacting NHS ester groups of the polymer with available amine groups on the protein. In a typical conjugation reaction, Cas9 nuclease and NHS-pCB at 1:10, 1:20, or 1:50 molar ratio were dissolved in 50 mM sodium borate buffer, pH 9.0. The final protein concentration was ~5 mg/mL. The reaction mixture was stirred for 2 hours at 4° C. and stopped by adjusting the pH of the mixture to 4.5 with glacialacetic acid. The polymer-protein conjugate was isolated via molecular weight cut-off (MWCO) spin dialysis membrane followed by ion-exchange chromatography. High performance liquid chromatography (HPLC) was used to measure the hydrodynamic size of the protein conjugates.

### 1.3 In Vitro Synthesis of sgRNA

The in vitro synthesis of sgRNA was carried out using EnGen® sgRNA Synthesis Kit using the manufacturer’s recommended conditions. The sgRNA product was purified using GeneJET RNA Cleanup and Concentration Micro Kit as described in the manual. The concentration of RNA was determined by measuring the absorbance at 260 nm on a microplate reader.

### 1.4 Mammalian Cell Culture

HEK 293-GFP cells were maintained in DMEM, medium supplemented with 10% FBS. U2OS cells were maintained in McCoy’s 5A modified medium supplemented with 25 mM HEPES and 10% FBS. K562 cells were propagated in RPMI 1640 medium containing 10% FBS. After thawing, cells were passaged 4-5 times before using for transfection. When setting up the experiments for transfections, cultured cells were plated in 24-well format (500 µl volume) in complete growth medium at a cell density necessary to reach ~70% confluence the next day. Full serum media was replaced with the same media but containing no antibiotics at least 1 h before delivery. All cultures were maintained in 5% CO2 at 37° C. in a humidified incubator.

### 1.5 In Vitro Co-Delivery of Cas9 Protein and sgRNA

For Cas9 protein transfection, 200 ng of purified Cas9 protein was added to 5 µl of Opti-MEM medium, followed by the addition of 50 ng gRNA. The molar ratio of gRNA to Cas9 protein was kept at approximately 1 to 1.2: 1. The sample was mixed by gently tapping the tubes a few times and then incubated at room temperature for 10 min. In a separate test tube, 0.8 µl of Lipofectamine CRISPRMAX transfection reagent was diluted to 5 µl with Opti-MEM medium. The diluted transfection reagent was transferred to the tube containing Cas9 protein/gRNA complexes, followed by incubation at room temperature for 10 min and then the entire solution was added to the cells in a 24-well plate and mixed by gently swirling the plate. The plate was incubated at 37° C. for 48 h in a 5% CO2 incubator.

### 1.6 Determination of On- and Off-Target Mutation Frequencies in Human Cells.

Genomic DNA was harvested 2 d after transfection from U2OS, HEK293 or K562 cells using the Quick-DNA Miniprep (Zymo Research), according to the manufacturer’s instructions. 100 ng of isolated genomic DNA was used as template to PCR amplify the targeted genomic sites with primer pairs. PCR products were purified with a PureLink™ PCR Purification Kit (Thermo Fisher) and quantified on a microplate reader. 250 ng of purified PCR DNA was combined with 2 µl of NEBuffer 2 (NEB) in a total volume of 19 µl and denatured then re-annealed with thermocycling at 95° C. for 5 min, 95-85° C. at 2° C./s; 85-20° C. at 0.2° C./s. The re-annealed DNA was incubated with 1 µl of T7 Endonuclease I (10 U/µl, NEB) at 37° C. for 30 min. Cas9-induced cleavage bands and the uncleaved band was visualized under UV light and quantified using ImageJ software30. The peak intensities of the cleaved bands were divided by the total intensity of all bands (uncleaved + cleaved bands) to determine the fraction cleaved, which was used to estimate gene modification levels. For each sample, transfections and subsequent modification measurements were performed in triplicate on different days. The Off-target analysis was performed using a bioinformatics-based search tool to select potential off-target sites, which was also evaluated using the T7E1 mutation detection assay.

### 1.7 Sanger Sequencing

To better determine the mutation rate, the same purified PCR products used for T7EI assay were sequenced to observe the individual mutations and determine the mutational spectra. Sanger sequencing was used to confirm the gene modification frequencies for the modified and unmodified CRISPR/Cas9 systems. The results were analyzed by ICE Analysis (Synthego).

### 1.8 Results

The “off-target” activity of the nucleases occurs fundamentally because the Cas9/sgRNA complex possesses more energy than what is needed for the effective recognition of its intended target DNA site. As a result, the complex lacks high specificity and is able to bind sequences that are similar to the on-target DNA strand. Therefore, the inventors hypothesized that the off-target effects of CRISPR/Cas9 might be minimized by reducing the non-specific interactions with its target DNA sites.

Zwitterionic poly(carboxybetaine) (pCB) polymers are highly hydrated and effectively resistant to non-specific interactions. Previously, pCB polymers have been conjugated to chymotrypsin (CT), uricase, and interferon-α2a to preserve protein bioactivity. The super-hydrophilic nature of the polymer creates an environment to shift the equilibrium and favor the substrate and the binding site to interact. It has been demonstrated that a pCB conjugated protein exhibits reduced non-specific interactions with its surrounding environment. The inventors have previously demonstrated that reduction of nonspecific interactions was shown to significantly enhance protein circulation time and reduce protein-specific anti-body production in vivo.

Since specific DNA-sgRNA matching is far stronger than non-specific interactions, the inventors hypothesized that pCB conjugation was able to reduce non-specific interactions and still maintain specific interactions strong enough with on-target DNA strands. With this strategy, the off-target effects of CRISPR/Cas9 can be minimized. Thus, an exemplary endonuclease, Cas9, was conjugated with pCB polymers and the on-target and off-target efficiency of the resulting conjugates has been examined. To assess the specificities of the pCB-conjugated CRISPR/Cas9 systems, a series of mismatched sgRNAs was designed, containing single, double, or triple substitutions within multiple sgRNA target DNA interfaces. Different endogenous human genes were tested. The exemplary modified endonucleases (e.g., pCB conjugates disclosed herein) showed decreased off-target activity compared with the unmodified (wild type) Cas9, but similar levels of on-target gene editing efficiency. Thus, modification of an RNA-guided endonuclease, such as Cas9, with a mixed charge moiety can provide a simple, safe, and robust strategy for CRISPR/Cas9 system-based gene editing.

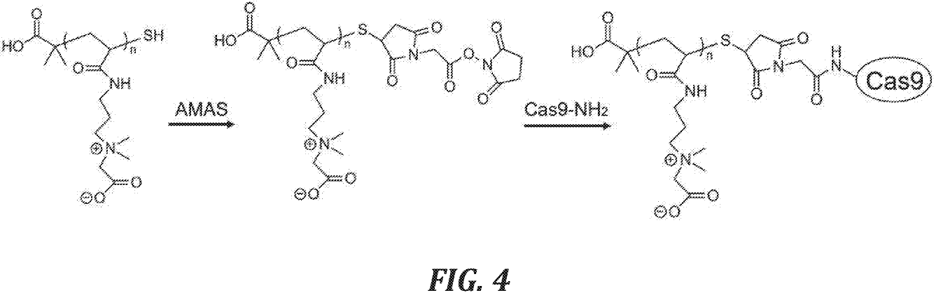

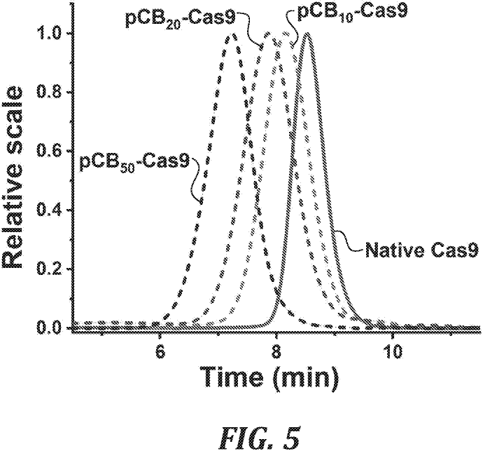

To examine the effect of the zwitterionic pCB polymer conjugation on the off-target efficiency of the CRISPR/Cas9 system, a series of exemplary pCB-Cas9 conjugates with different numbers of polymer chains per protein were prepared. Conjugates of pCB-Cas9 were synthesized by reacting N-Hydroxysuccinimide (NHS) ester groups of the polymer with available amine groups on the protein. The reaction scheme is illustrated in FIG. 5. The polymer density was controlled by altering the molar ratio between Cas9 and NHS-pCB in the reaction. Exemplary modified endonucleases denoted pCB10-Cas9, pCB20-Cas9, and pCB50-Cas9 were synthesized at the molar ratio of 1:10, 1:20, and 1:50, respectively. A native (unconjugated) Cas9 protein was used for comparison throughout the disclosure. The difference in size between native Cas9 and pCB-Cas9 conjugates is shown in FIG. 6B, which confirms the successful synthesis of the polymer-protein conjugates.

In almost all cases, surface modification of a protein by covalent conjugation with polymers, such as PEGylation, lowers the in vitro biological activity of conjugated proteins. Therefore, the inventors first tested whether the presence of zwitterionic polymers would compromise the on-target activity after conjugation. For these experiments, a well-established Cas9-induced GFP disruption assay was used that enabled the rapid quantification of targeted nuclease activities (Y. Fu, J. A. Foden, C. Khayter, M. L. Maeder, D. Reyon, J. K. Joung, J. D. Sander, Nature biotechnology 2013, 31, 822, the disclosure of which is incorporated herein by reference).

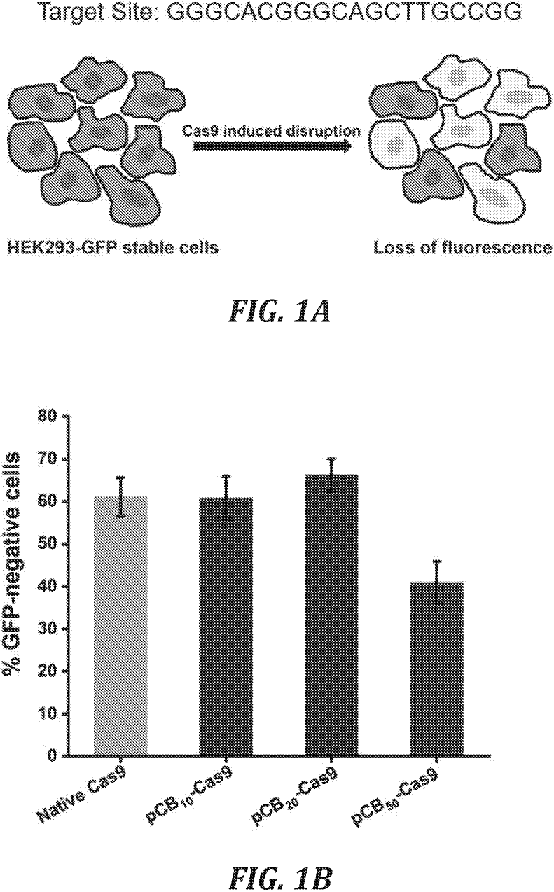

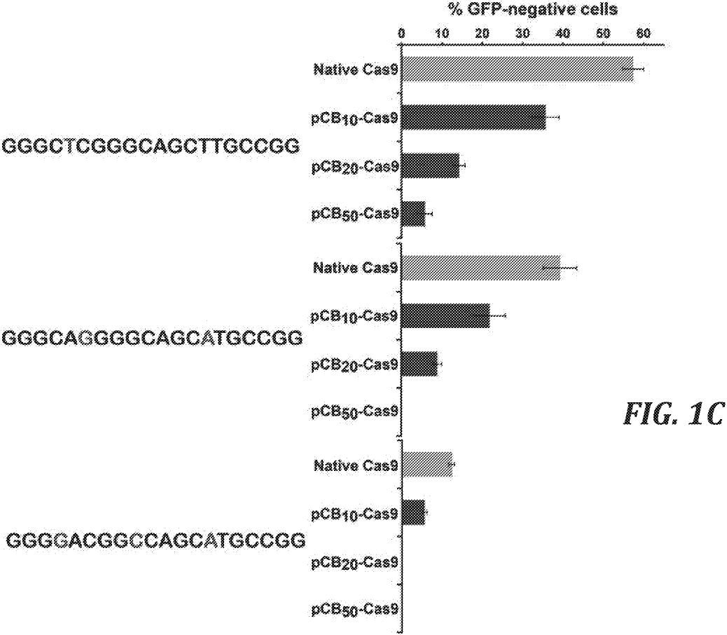

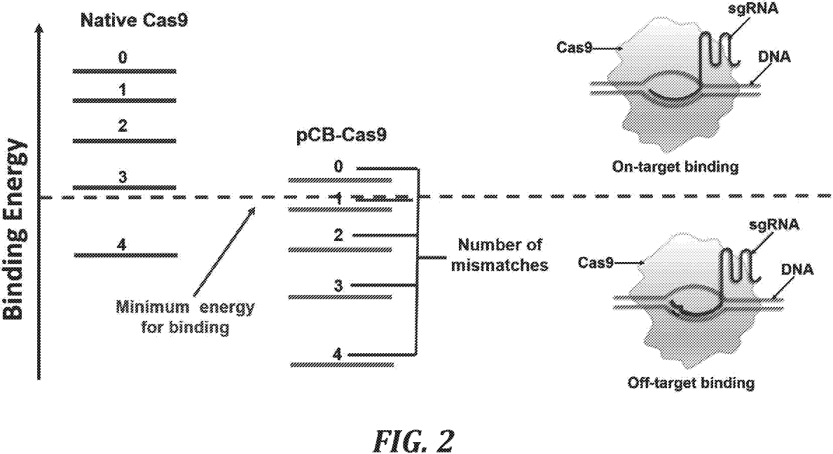

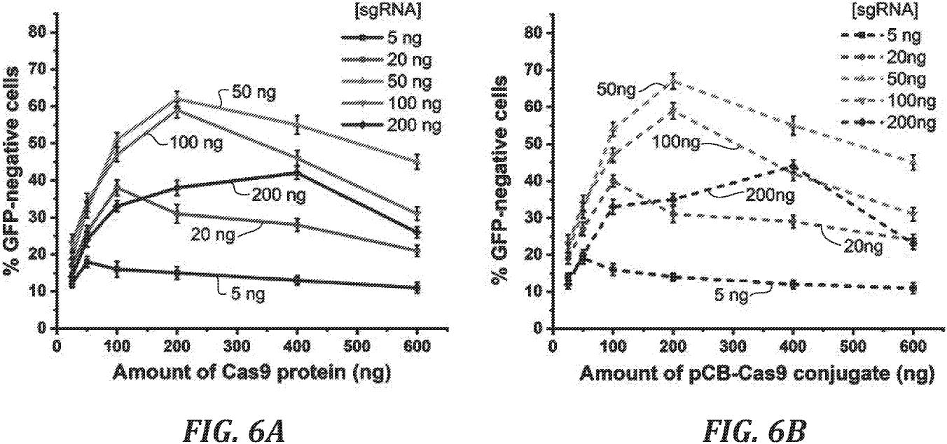

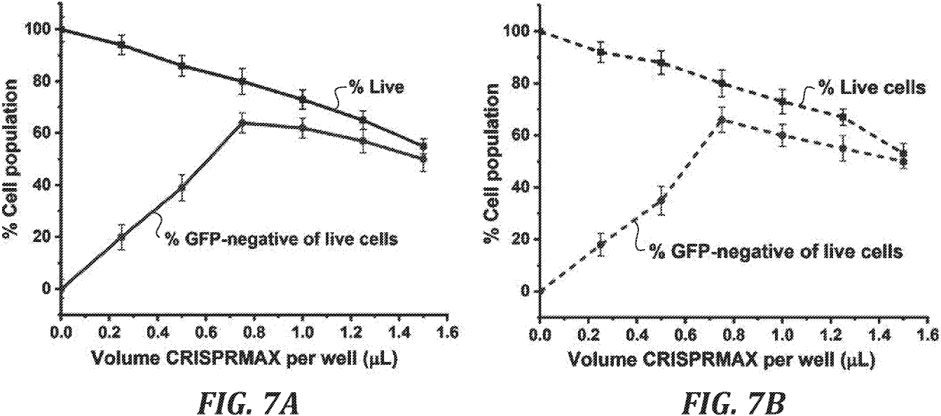

In this assay, a genomic GFP reporter gene in human HEK293-GFP cells was targeted. The activities were quantified by measuring the loss of fluorescence signal in human HEK293-GFP cells, which is caused by the on-target CRISPR/Cas9 cleavage (FIG. 1A). The cells were treated with 50 ng sgRNA and 200 ng native Cas9/Cas9-equivalent pCB-Cas9 conjugates with CRISPRMAX (FIGS. 7 and 8) in DMEM containing 10% FBS for 48 hours to induce the disruption of GFP reporter gene. As shown in FIG. 1B, it was found that exemplary endonucleases pCB₁₀-Cas9 and pCB₂₀-Cas9 showed comparable editing efficiency to native Cas9, in which about 60% cells lost their GFP expression after the treatment. The results demonstrated that the presence of pCB polymer did not compromise the “on-target” editing efficiency of the CRISPR/Cas9 system. However, only 40% GFP negative cells were found when treated with pCB₅₀-Cas9 (FIG. 1B). This was due to the presence of more pCB polymers, which can physically impede the binding between DNA and CRISPR/Cas9 RNP.

To explore the potential of pCB conjugates in reducing the off-target activity, variant sgRNAs for the target site with one, two, or three mismatched nucleotides were randomly generated and tested whether these mismatched sgRNAs could drive off-target GFP disruption in human cells (FIG. 1C). If pCB conjugation could reduce off-targeting, then pCB-Cas9 conjugates would be less tolerant of mismatches than native Cas9. As presented in FIG. 1C, native Cas9 can still induce substantial GFP gene disruption in human cells when using mismatched sgRNA. In contrast, pCB-Cas9 conjugates showed a significant reduction of GFP disruption efficiency when mismatched sgRNAs were used (FIG. 1C). pCB₁₀-Cas9 induced 35.6%, 21.9%, and 5.6% GFP disruption while pCB₂₀-Cas9 led to 14.2%, 8.9%, and 0%, respectively when one, two, or three nucleotide mismatches were present in the sgRNA. pCB₅₀-Cas9 generated no detectable GFP disruption when 2 or 3 nucleotide mismatches were present. These data suggest that pCB conjugation can significantly reduce the off-target gene editing when mismatched sgRNAs are used. Taking both the on-target and off-target efficiency into consideration, pCB₂₀-Cas9, which shows complete on-target efficiency and significantly reduced off-target efficiency, was selected for further studies described below.

To further evaluate the effects of mismatches on pCB-Cas9, additional sgRNA bearing one, two, or three nucleotide mutations were designed. As shown in Table 1, native Cas9 exhibited significant off-target editing in a single-mismatch scenario (57.4%, 60.6%, and 49.3%). These off-target editing efficiencies are very close to its on-target editing efficiency in the perfect-match scenario (62.6%) as discussed above. In contrast, using the same mismatched sgRNAs, exemplary pCB₂₀-Cas9 conjugate showed a significantly reduced off-target editing efficiency (14.2%, 12.6%, and 6.8%). This editing efficiency is more than 80% lower than its editing efficiency in the perfect-match scenario (67.4%). This observation was confirmed in another two scenarios when the sgRNAs had two or three mismatches. When 3 mismatches are presented in a sgRNA, the native Cas9 still exhibited positive editing efficiencies (12.4%, 7.9%, 9.3%). In contrast, no “off-target” efficiency is observed for pCB-Cas9 conjugate groups. This indicates that pCB-conjugated CRISPR/Cas9 has a high resolution in DNA editing and can distinguish the mismatch on a single-base level.

On-target and known off-target substrates of Cas9:sgRNAs that target sites

in GFP, EMX, VEGF, and CLTA. List of genomic on-target and off-targets sites for GFP, EMX, VEGF, and CLTA are shown with mutations from the on-target sequence shown in lower case.

TABLE 1

| SEQ ID NO: | Target | Site name | Sequence |
|---|---|---|---|
| 5 | Target 1 (GFP) | GFP-On | GGGCACGGGCAGCTTGCCGG |
| 6 | Target 2 (VEGF) | VEGF-On | GGGTGGGGGGAGTTTGCTCC |
| 7 | | VEGF-Off1 | GGaTGGaGGGAGTTTGCTCC |
| 8 | | VEGF-Off2 | GGGaGGGtGGAGTTTGCTCC |
| 9 | | VEGF-Off3 | cGGgGGaGGGAGTTTGCTCC |
| 10 | | VEGF-Off4 | GGGgaGGGGaAGTTTGCTCC |
| 11 | Target 3 (EMX) | EMX-On | GAGTCCGAGCAGAAGAAGAA |
| 12 | | EMX-Off1 | GAGgCCGAGCAGAAGAAagA |
| 13 | | EMX-Off2 | GAGTCCtAGCAGgAGAAGAA |
| 14 | | EMX-Off3 | GAGTCtaAGCAGAAGAAGAA |
| 15 | | EMX-Off4 | GAGTtaGAGCAGAAGAAGAA |
| 16 | Target 4 (CLTA) | CLTA-On | GCAGATGTAGTGTTTCCACA |
| 17 | | CLTA-Off1 | aCAtATGTAGTaTTTCCACA |
| 18 | | CLTA-Off2 | cCAGATGTAGTaTTcCCACA |
| 19 | | CLTA-Off3 | ctAGATGaAGTGcTTCCACA |
| 20 | | CLTA-Off4 | ctAGATGaAGTGcTTCCACA |

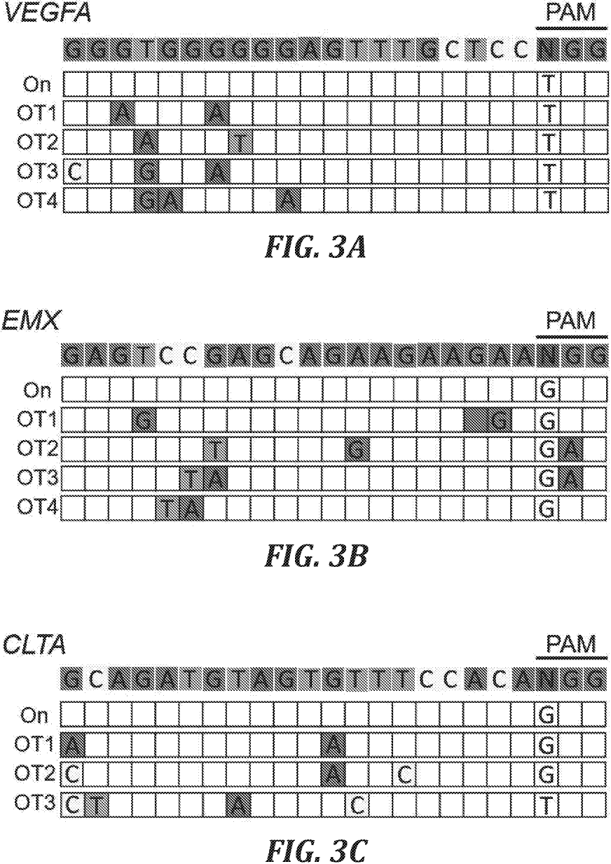

It is known that the “off-target” activity of the nucleases is fundamentally caused by the extra energy that the Cas9/sgRNA complex possesses, leading to the lack of perfect specificity. Such extra energy comes mainly from the nonspecific forces -hydrophobic and electrostatic in particular. Coating a protein with non-fouling polymeric materials can alter these nonspecific interactions and is the key to lower energy and promote specific binding. It is known that the “off-target” activity of the nucleases is fundamentally caused by the extra energy that the Cas9/sgRNA complex possesses, leading to the lack of perfect specificity. Such extra energy comes mainly from the nonspecific forces - hydrophobic and electrostatic in particular. Coating a protein with non-fouling polymeric materials can alter these nonspecific interactions and is the key to lower energy and promote specific binding. Over the last several years, zwitterionic materials based on naturally occurring betaines such as pCB have particularly high hydration. As a result, ultra-low nonspecific adsorption in complex biological media has been observed in different scenarios. The inventors hypothesized that the conjugation of pCB to Cas9 could reduce the nonspecific binding force between the Cas9/sgRNA complex and the target DNA. As shown in FIG. 2, for native Cas9, the energy that the Cas9/sgRNA complex possesses is much higher than the minimum energy required for on-target binding between the sgRNA and the DNA. As a result, the RNP complex still possesses enough energy even one or more mismatched nucleotides are present. With the pCB conjugates, the aforementioned nonspecific binding, especially the hydrophobic-hydrophobic interaction, is decreased significantly. The complex is unable to bind the double-strand DNA without sufficient energy when mismatched nucleotides are present on the sequence. As a result, off-target effects can be reduced significantly. In addition, benefiting from the super-hydrophilicity of the polymer, a tightly bound water layer is formed around and the nonspecific interactions between RNPs and zwitterionic polymers can be minimized as observed before. As a result, the bioactivity of Cas9 can be retained after the polymer conjugation, which is very important to keep the on-target efficiency.

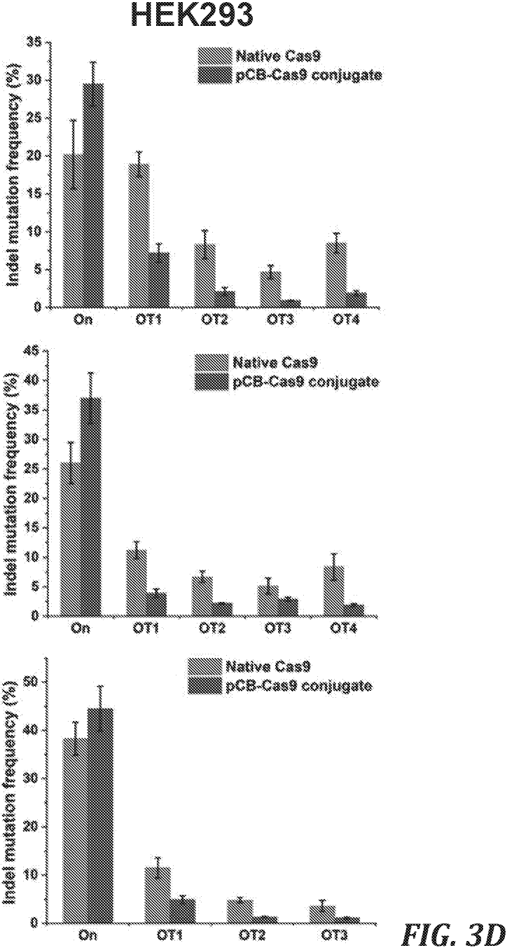

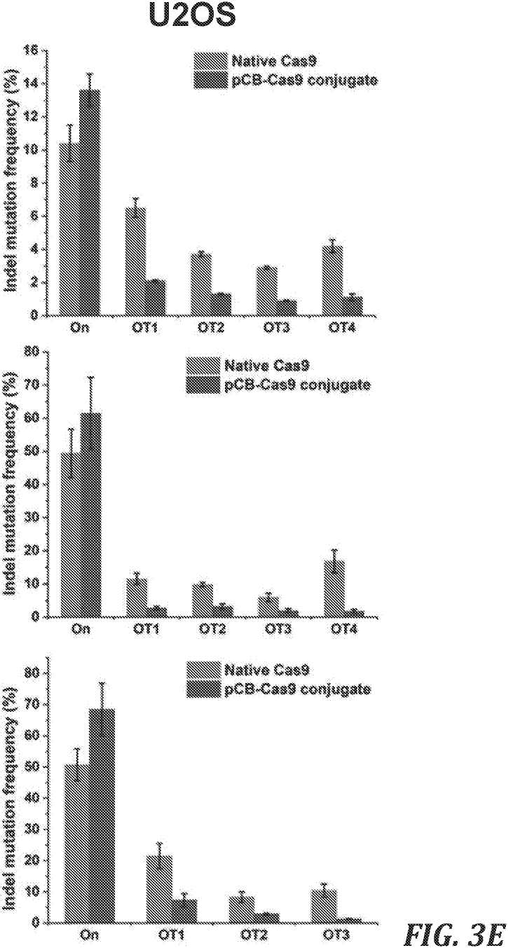

To examine whether pCB-conjugated CRISPR/Cas9 RNPs can reduce off-target effects on other DNA domains in human cells, three new genomic loci in the VEGFA, EMX, and CLTA genes were selected due to their potential biomedical relevance and widely use in Cas9 off-target studies. As presented in FIG. 3D, for all three targets, CRISPR/pCB₂₀-Cas9 mediated indels at their endogenous loci were detected using the T7 endonuclease I (T7EI) assay. For each of these three target sequences, the inventors examined the editing efficiencies of several potential off-target sites which have been observed in other studies. In this disclosure, similar trends were observed. The rates of mutation at the selected off-target sites were very high, ranging from 9.4% to 93.6% when the cells were edited using native CRISPR/Cas9. In contrast, for the cells edited using exemplary pCB₂₀-conjugated CRISPR/Cas9, the off-target mutation rates were observed at a much lower level, ranging from 2.4% to 10.5%. It is noticeable that the editing efficiency of the pCB-Cas9 conjugate is slightly higher than that of native Cas9. This confirms the hypothesis that the bioactivity of Cas9 is preserved after conjugation.

After demonstrating that pCB conjugation reduced the off-target mutations of CRISPR/Cas9 in HEK293-GFP cells, this modified Cas9 was evaluated in other types of human cells. Here, U2OS and K562 cell lines were used, as they were also widely used to test the on-/off-target activity of CRISPR/Cas9. The editing efficiencies on three targets were explored using either native Cas9 or pCB-Cas9 in U2OS and K562 cell editing. As expected, pCB-Cas9 conjugates generate similar or slightly higher editing efficiency when compared with native Cas9 at the target genomic locus (FIGS. 3E and 3F). For the off-target examinations using variant mismatches, the pCB-Cas9 conjugate groups produced less than 4% indels rates at 20 out of 22 off-target sites. In contrast, native Cas9 generated 2.9% - 24.7% off-target indels, of which six were higher than 10%. These results further demonstrated that pCB-conjugated CRISPR/Cas9 showed reduced off-target effects in different cell lines.

### Example 2: Preparation of an Exemplary Modified Endonuclease (Fusion Protein Cas9-EK) and Ex Vivo Cas9-EK mRNA Delivery for Gene Editing and Off-Targeting Reduction.

In this example, human codon-optimized DNA encoding Cas9 nuclease from Streptococcus pyogenes with N and C terminal nuclear localization signal (NLS) was cloned into a pcDNA3.1 vector. DNA encoding poly(EK) with 10 kDa or 30 kDa length were commercially synthesized and appended to the C-terminal or both C- and N-terminals of the Cas9 gene to generate Cas9-EK constructs (FIGS. 8A-8C). 3 Cas9-EK plasmids, Cas9-(EK)₁₀, (EK)₁₀-Cas9-(EK)₁₀, and Cas9-(EK)₃₀, based on the length and fusion position of poly(EK) were constructed. The Cas9 sequence without poly(EK) sequence was used as the control sequence. Since the constitutive presence of the plasmids and transcripts could result in high levels of undesired off-target gene editing, DNA-free CRISPR gene-editing systems were used by transfecting both in vitro transcribed (IVT) sgRNA and Cas9 mRNA to achieve their desired gene editing effect. IVT mRNA minimizes the risk of genome insertion, and it bypasses the requirement of nuclear entry for transcription, resulting in quick onset of genome editing. In addition, Cas9 mRNA delivery provides a transient expression of Cas9 protein, which may potentially decrease off-target effects.

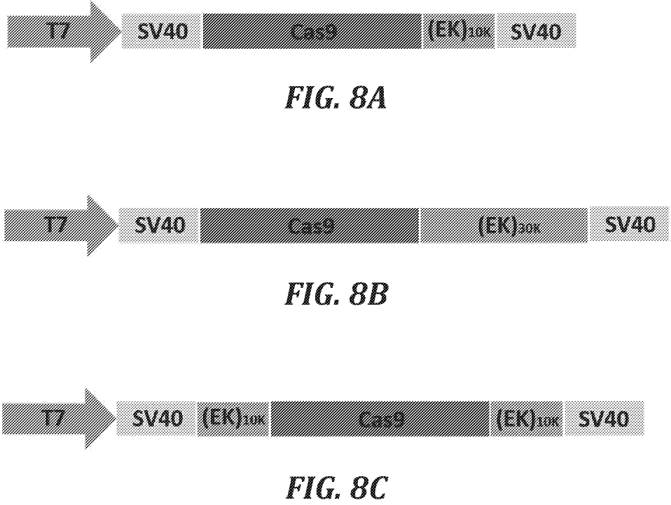

Cas9 and Cas9-EK mRNAs were generated through in vitro transcription. Cas9 and Cas9-EK encoding plasmids were linearized using BbsI (New England Biolabs) according to the manufacturer’s instructions. Following purification, the Cas9 and Cas9-EK mRNAs were transcribed using mMESSAGE mMACHINE® T7 Ultra Transcription Kit (ThermoFisher) according to manufacturer’s instructions with a 2-hour incubation time at 37° C. TURBO DNase was added to stop transcription. This system relies on the translation of Cas9 mRNA in cells, so polyadenylation (poly(A)) of Cas9 and Cas9-EK mRNA prior to transfection is required to prevent Cas9 mRNA from degradation before in vivo translation occurs. The polyadenylation reaction was started with the addition of the E-PAP enzyme and incubated for 30 mins at 37° C. The band shift after polyadenylation in the electrophoresis image (FIG. 9) confirmed the presence of poly(A) tails. Smeared bands indicated degradation.

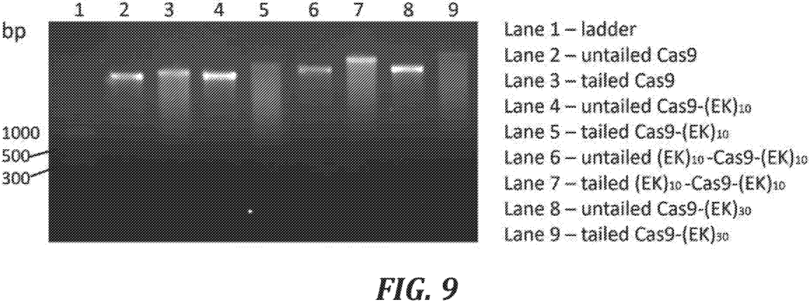

After getting the mRNA, the activity of lab prepared Cas9 mRNA in mammalian cells was first confirmed by comparing it with the commercialized Cas9 mRNA. The gRNA that target GFP (SEQ. ID NO: 5) was selected for this analysis. One day prior to transfection, the cells were seeded in a 24-well plate at a cell density of 1-2 × 10⁵ cells per well. 0.5 µg Cas9 or Cas9-EK mRNA was added to 25 µL of Opti-MEM, followed by the addition of 50-100 ng gRNA. Meanwhile, 2 µL of Lipofectamine MessengerMax (ThermoFisher) was diluted into 25 µL of Opti-MEM and then mixed with mRNA/gRNA sample. The mixture was incubated for 15 minutes prior to addition to the cells. Then the entire solution was added to the cells and mixed by gently swirling the plate. The plate was incubated at 37° C. for 48 h in a 5% CO₂ incubator. Two days after co-transfecting HEK293-GFP cells with mRNA expressing either commercialized Cas9 or lab-prepared Cas9 together with the GFP gRNA, the percentage of indel mutations was quantified by the T7EI assay. As shown in FIG. 10, the lab-prepared Cas9 mRNA possesses similar on-target editing efficiency to the commercial Cas9 mRNA, which indicates the successful synthesis of the in vitro transcribed Cas9 mRNA.

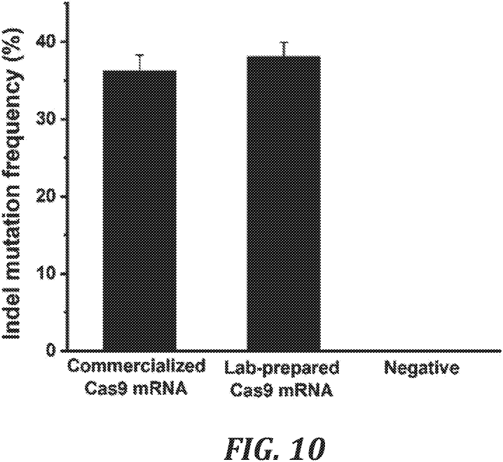

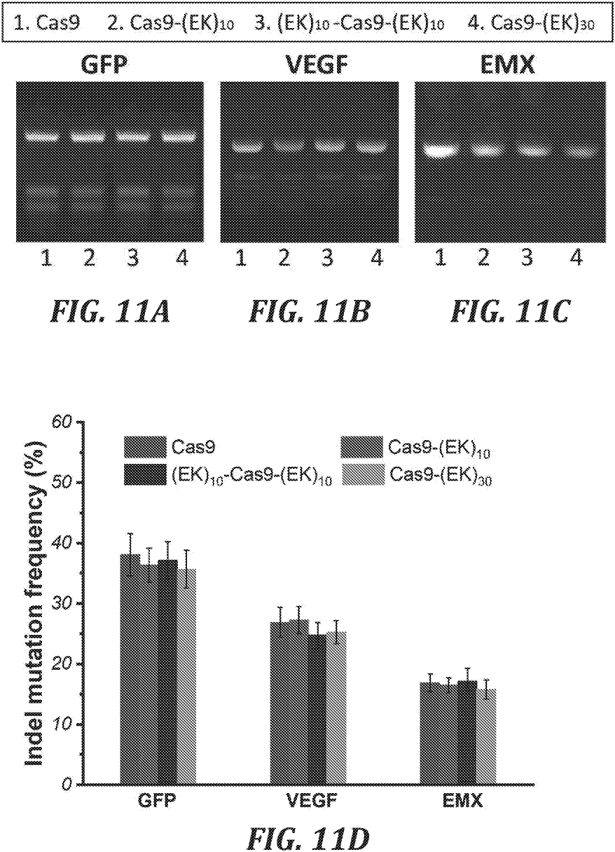

To investigated whether the Cas9-EK mRNAs could be programmed by gRNAs to cleave chromosomal DNA in mammalian cells, the same assay was used to test the gene-editing efficiency of Cas9-(EK)_{10,} (EK)₁₀-Cas9-(EK)₁₀, and Cas9-(EK)₃₀. As shown in FIG. 11A, all three Cas9-EK mRNAs show a similar editing level to the Cas9 mRNA when targeting the GFP sequence. The presence of poly(EK) did not compromise the on-target gene editing efficiency on the selected on-target site. To approve that this effect is not targeting site-specific, the similar editing frequency was further verified with genomic loci VEGF (GGGTGGGGGGAGTTTGCTCC) (FIG. 11B) and EMX (GAGTCCGAGCAGAAGAAGAA) (FIG. 11C). The quantified data are summarized and presented in FIG. 11D. All three Cas9-EK fusions show similar level of on-target editing efficiency to native Cas9 mRNA for both VEGF and EMX target loci.

To examine whether the presence of poly(EK) can reduce the off-target effects in human cells, several potential off-target sites for VEGF and EMA target loci were selected, which have been observed in other studies. The results are shown in Table 2. For Cas9-(EK)₁₀, the off-target activity was similar or slightly reduced for some off-target sites compared to the native Cas9. Without wishing to be bound by theory, it is believed that this is due to the insufficient length of poly(EK) that can’t reduce the nonspecific force between gRNA and double-strand DNA. However, both (EK)₁₀-Cas9-(EK)₁₀ and Cas9-(EK)₃₀ showed a significantly reduced off-target editing efficiency, ranging from 1.2% to 3.5%. These results demonstrated that Cas9-EK showed reduced off-target effects on different genomic loci.

TABLE 2

On-target and off-target DNA modification resulting from native CRISPR/Cas9 and Cas9-EK fusions in HEK293-GFP, U2OS and K562 cells. (N.D., none detected)

| SEQ ID NO: | Indel mutation frequency (%) | | | | | | | | | | | |
|---|---|---|---|---|---|---|---|---|---|---|---|---|
| | HEK293 | | | | U2OS | | | | K562 | | | |
| | Cas9: (%) | Cas9-(EK)₁₀ (%) | (EK)₁₀- Cas9-(EK)₁₀ (%) | Cas9-(EK)₃₀ (%) | Cas9 (%) | Cas9-(EK)₁₀ (%) | (EK)₁₀ -Cas9-(EK)₁₀ (%) | Cas9-(EK)₃₀ (%) | Cas9 (%) | Cas9-(EK)₁₀ (%) | (EK)₁₀ -Cas9-(EK)₁₀ (%) | Cas9-(EK)₃₀ (%) |
| 6 | 26.9 | 27.3 | 24.8 | 25.3 | 34.6 | 32.5 | 32.1 | 30.6 | 11.5 | 11.2 | 10.4 | 9.7 |
| 7 | 7.4 | 7.0 | 3.5 | 1.7 | 21.5 | 19.4 | 5.8 | 4.9 | 6.3 | 6.1 | 1.4 | 1.5 |
| 8 | 8.5 | 7.6 | 2.6 | 3.7 | 8.9 | 8.1 | 2.3 | 1.9 | 3.4 | 3.1 | N.D. | N.D. |
| 9 | 6.8 | 4.9 | 2.1 | 1.9 | 6.4 | 5.8 | 1.2 | 1.1 | 2.5 | 1.9 | N.D. | N.D. |
| 10 | 4.5 | 4.8 | 1.2 | 1.5 | 10.4 | 9.1 | 2.1 | 1.6 | 1.5 | 1.9 | N.D. | N.D. |
| 11 | 16.9 | 16.5 | 17.2 | 15.8 | 43.6 | 42.7 | 41.9 | 40.1 | 14.7 | 13.8 | 12.2 | 11.8 |
| 12 | 7.5 | 7.0 | 2.4 | 2.1 | 12.4 | 11.6 | 3.6 | 2.8 | 3.8 | 4.0 | 1.3 | N.D. |
| 13 | 5.7 | 5.9 | 1.9 | 2.1 | 10.5 | 10.4 | 3.7 | 3.1 | 3.1 | 2.6 | N.D. | N.D. |

After poly(EK) fusion has been demonstrated to reduce the off-target mutations of CRISPR/Cas9 in HEK293-GFP cells, this EKylated Cas9 was evaluated in other types of human cells. Here, U2OS and K562 cell lines were employed as they were also widely used to test the on-/off-target activity of CRISPR/Cas9. The editing efficiencies on two targets were explored, using either native Cas9 or Cas9-EK in U2OS and K562 cell editing. As expected, Cas9-EK fusions generate similar on-target editing efficiency when compared with native Cas9 at the target genomic locus (Table 2). For the off-target examinations using variant mismatches, Cas9-(EK)₁₀ shows similar off-target gene editing efficiency to native Cas9. However, (EK)₁₀-Cas9-(EK)₁₀ and Cas9-(EK)₃₀ showed a significantly reduced off-target editing efficiency produced less than 3% indels rates at 29 out of 36 off-target sites. In contrast, native Cas9 mRNA generated 2.5% -21.5% off-target indels, of which four were higher than 10%. These results further demonstrated that poly(EK) fused CRISPR/Cas9 showed reduced off-target effects in different cell lines.

### Example 3: Ex-Vivo and in Vivo mRNA Delivery of CRISPR/Cas9 System Fused with Mixed Charge Polypeptide.

The efficacy of the exemplary system can be further demonstrated in an ex vivo system. The above-described protocol can be used for the gene editing of primary cells with mutations. For example, re-expression of the paralogous γ-globin genes (HBG1/2) could be a universal strategy to ameliorate the severe β-globin disorders sickle cell disease (SCD) and β-thalassemia by induction of fetal hemoglobin (HbF, α2γ2). It has been known that core sequences at the BCL11A erythroid enhancer are required for repression of HbF in adult stage erythroid cells but are dispensable in non-erythroid cells. Cas9:sgRNA-mediated cleavage within a GATA1 binding site at the BCL11A erythroid enhancer can result in highly penetrant disruption of this motif, reduction of BCL11A expression, and induction of fetal γ-globin. The experiments are performed with human adult hematopoietic stem and progenitor cells (HSPCs) harvested from SCD patients or β-thalassemia patients. The Cas9/Cas9-(EK)ₙ mRNA and sgRNA (SEQ. ID NO: 21-24) is delivered as described above. The editing efficiency is assessed by T7E1 assays, site-specific Sanger sequencing, and deep sequencing of on-target and putative off-target sites. The HSPCs is expected to preferentially undergo non-homologous end joining repair. Erythroid progeny of edited engrafting SCD HSCs is expected to express therapeutic levels of HbF and resist sickling, while those from patients with β-thalassemia is expected to show restored globin chain balance. After edited with CRISPR/Cas9 system, the human CD34⁺HSPCs are injected into the immunodeficient NOD.Cg-Kit^{W-41J} Tyr⁺ Prkdc^{scid} I12rg^{tm1wj1} (NBSGW) mice to test the impact of BCL11A enhancer editing on HSPCs. The Non-irradiated NBSGW female mice (4-5 weeks of age) is infused by retro-orbital injection with 0.2-0.8 M CD34⁺ HSPCs resuspended in DPBS. Bone marrow is isolated for human xenograft analysis 16 weeks post-engraftment. Serial transplants are conducted using retro-orbital injection of bone marrow cells from the primary recipients. For flow cytometry, analysis of bone marrow cells is carried out to measure the percentage human engraftment.

### Example 4: In Vivo mRNA Delivery of CRISPR/Cas9 System Fused With Mixed Charge Polypeptide.

CRISPR/Cas9 gene editing can be accomplished by transformation of DNA plasmid encoding both Cas9 and sgRNA, but the constitutive presence of the plasmids and transcripts can result in high levels of undesired off-target gene editing. Many researchers are turning to DNA-free CRISPR gene editing systems by transfecting both in vitro transcribed sgRNA and Cas9 mRNA to achieve their desired gene editing effect. In this example, human codon-optimized DNA encoding Cas9 nuclease from Streptococcus pyogenes with an N and C terminal nuclear localization signal (NLS) is cloned into a pcDNA3.1 vector (GenScript). DNA sequence encoding poly(EK) with 10 KDa or 30 KDa length is commercially synthesized and appended to the 3′-terminal or both 5′- and 3′-terminals of the Cas9 gene to generate Cas9-EK constructs. gRNA is identified by searching for an on-target sequence within mouse Pcsk9 exons that showed a high number of off-target sites (two or fewer mismatches to the on-target site) in the mouse genome. Cas9 and Cas9-EK mRNAs is generated through in vitro transcription using mMESSAGE mMACHINE® T7 Ultra Transcription Kit (ThermoFisher) according to manufacturer’s instructions. The Cas9 or Cas9-EK mRNA and gRNA is encapsulated in a lipid nanoparticle (LNP) for systemic delivery.

For in vivo Pcsk9 gene editing, nine- to eleven-week-old male mice receive a tail vein injection with consistent ratio of Cas9 to gRNA with phosphate-buffered saline. Peripheral blood is sampled before mRNA administration (baseline), a week after virus administration and at termination (four days or three weeks after mRNA administration). Animals are euthanized by cardiac puncture under isoflurane anesthesia at the experimental endpoint. The organs—including liver, spleen, lungs, kidney, muscle, brain and testes-are dissected, snap-frozen in liquid nitrogen and stored at -80° C. until further analyses. Peripheral blood is collected in EDTA-coated capillary tubes from vena saphena during the course of the study and by cardiac puncture at the time of termination. Levels of mouse Pcsk9 in plasma are determined with a standard ELISA kit according to the manufacturer’s instructions. Genomic DNA from liver tissue of adenovirus-injected mice is extracted at day 4 and at week 3 post-treatment for indel analysis. The on-target site and a various of identified potential off-target sites are analyzed by deep sequencing.

In summary, the above examples demonstrate that the “off-target” concern faced by the CRISPR/Cas system can be addressed by modification with mixed charge moieties. After being modified with a mixed charge polymer or a mixed charge peptide, exemplary CRISPR/Cas9 system showed significantly reduced “off-target” efficiency. More importantly, no reduced “on-target” frequency, which is usually noticed in many strategies, was observed. This technology can provide a simple and robust strategy to improve the efficiency and safety of a wide range of CRISPR/Cas9-based biological and clinical applications.

While illustrative embodiments have been illustrated and described, it will be appreciated that various changes can be made therein without departing from the spirit and scope of the invention.

1. A modified endonuclease, comprising an endonuclease and one or more mixed charge moieties covalently linked to the endonuclease, wherein each mixed charge moiety comprises about 10 to about 400 positively charged moieties and about 10 to about 400 negatively charged moieties, and wherein the ratio of the number of positively charged moieties to the number of negatively charged moieties is from about 1:0.5 to about 1:2.

2. The modified endonuclease of claim 1, wherein the endonuclease is a nucleic acid-guided nuclease system protein.

3. The modified endonuclease of any one of claims 1 or 2, wherein the endonuclease is a CRISPR-associated (Cas) protein.

4. The modified endonuclease of any one of claims 1-3, wherein the endonuclease is Cas9, Cas12, Cas13, Cas14, or a mutant or a variant thereof.

5. The modified endonuclease of any one of claims 1-4, wherein the endonuclease is Cas9 or a mutant or a variant thereof.

6. The modified endonuclease of any one of claims 1-5, wherein the mixed charge moiety is substantially electronically neutral at pH of about 7.4.

7. The modified endonuclease of any one of claims 1-6, wherein the endonuclease is active in a CRISPR/Cas system, wherein the CRISPR/Cas system displays reduced off-target editing activity and maintained on-target editing activity relative to a wild-type CRISPR/Cas system.

8. The modified endonuclease of claim 7, wherein the off-target editing activity is reduced by at least 20%, at least 25%, at least 30%, at least 35%, at least 40%, at least 45%, at least 50%, at least 55%, at least 60%, at least 65%, at least 70%, at least 75%, or at least 80% compared to an unmodified endonuclease.

9. The modified endonuclease of any one of claims 1-8 wherein the mixed charge moiety is covalently linked to a side chain of an amino acid of the endonuclease, to the N-terminal amino group of the endonuclease, and/or to the C-terminal carboxylic group of the endonuclease.

10. The modified endonuclease of any one of claims 1-9, wherein the mixed charge moiety is a peptide with a molecular weight of about 2 kDa to about 130 kDa.

11. The modified endonuclease of claim 10, wherein the modified endonuclease is a fusion protein, wherein the mixed charge moiety is a mixed charge domain consisting of:
a) a plurality of negatively charged amino acids;
b) a plurality of positively charged amino acids; and
c) optionally a plurality of additional amino acids independently selected from the group consisting of proline, serine, threonine, asparagine, glutamine, glycine, and derivatives thereof; and
wherein the ratio of the number of positively charged amino acids to the number of negatively charged amino acids is from about 1:0.5 to about 1:2.

12. The modified endonuclease of claim 10, wherein the plurality of negatively charged amino acids is independently selected from the group consisting of aspartic acid, glutamic acid, and derivatives thereof.

13. The modified endonuclease of claim 10, wherein the plurality of positively charged amino acids is independently selected from the group consisting of lysine, histidine, arginine, and derivatives thereof.

14. The modified endonuclease of claim 10, wherein the mixed charge domain does not comprise a plurality of additional amino acids.

15. The modified endonuclease of claim 10, wherein the plurality of positively charged amino acids are lysines and a plurality of negatively charged amino acids are glutamic acids.

16. The modified endonuclease of claim 10, wherein the mixed charge domain comprises a random sequence.

17. The modified endonuclease of claim 10, wherein the mixed charge domain comprises a sequence (X1-X2-X3)ₙ, wherein X1 is a positively charged amino acid, X2 is a negatively charged amino acid, and X3 is absent or is an additional amino acid independently selected from the group consisting of proline, serine, threonine, asparagine, glutamine, glycine, and derivatives thereof, wherein n is an integer from about 5 to about 50.

18. The modified endonuclease of claim 10, wherein the mixed charge domain comprises a plurality of lysines and a plurality of negatively charged amino acids selected from the group consisting of glutamic acid and aspartic acid.

19. The modified endonuclease of claim 10, wherein the mixed charge domain comprises a plurality of histidines and a plurality of negatively charged amino acids selected from the group consisting of glutamic acid and aspartic acid.

20. The modified endonuclease of claim 10, wherein the plurality of additional amino acids is selected from the group consisting of proline, serine, and glycine.

21. The modified endonuclease of claim 10, wherein the plurality of additional amino acids is a plurality of prolines.

22. The modified endonuclease of claim 10, wherein the mixed charge domain comprises a plurality of lysines, a plurality of glutamic acids, and a plurality of prolines.

23. The modified endonuclease any one of claims 1-9, wherein the mixed charge moiety is a synthetic polymer with a molecular weight of about 2 kDa to about 80 kDa.

24. The modified endonuclease of claim 23, wherein the polymer selected from the group consisting of poly(carboxybetaine) (PCB), poly(sulfobetaine) (PSB), poly(2-methacryloyloxyethyl phosphorylcholine) (PMPC), and poly(trimethylamine oxide) (TMAO) polymers.

25. The modified endonuclease of any one of claims 23 or 24, wherein the polymer is a poly(carboxybetaine) (PCB).

26. A nucleic acid comprising a sequence encoding the modified endonuclease of any one of claims 1-22.

27. An expression vector comprising the nucleic acid of claim 26 and a promoter operably linked thereto.

28. A cell comprising the nucleic acid of claim 26 or the expression vector of claim 27.

29. The cell of claim 28, wherein the cell is a prokaryotic cell or eukaryotic cell.

30. The cell of claim 28, wherein the cell is a mammalian cell.

31. The cell of claim 28, wherein the cell is in a cell culture.

32. The cell of claim 28, wherein the cell is in an organism.

33. A method for editing a polynucleotide in a cell or in a subject, the method comprising introducing into the cell or the subject at least one modified endonuclease of any one of claims 1-25, a nucleic acid of claim 26, or an expression vector of claim 27.

34. The method of claim 33, wherein the polynucleotide is DNA or RNA.

35. The method of claim 33 or 34, wherein the nucleic acid is an mRNA encoding the modified endonuclease.

36. The method any one of claims 33-35, wherein the modified endonuclease comprises a mixed charge moiety covalently linked to an endonuclease, wherein the mixed charge moiety comprises about 10 to about 400 positively charged moieties and about 10 to about 400 negatively charged moieties, and wherein the ratio of the number of positively charged moieties to the number of negatively charged moieties is from about 1:0.5 to about 1:2.

37. The method of any one of claims 33-36, wherein the endonuclease is a nucleic acid-guided nuclease system protein.

38. The method of any one of claims 33-37, wherein the endonuclease is a CRISPR-associated (Cas) protein.

39. The method of any one of claims 33-38, wherein the endonuclease is Cas9, Cas12, Cas13, Cas14, or a mutant or a variant thereof.

40. The method of any one of claims 33-39, wherein the endonuclease is Cas9 or a mutant or a variant thereof.

41. The method of any one of claims 34-40, wherein the mixed charge moiety is substantially electronically neutral at pH of about 7.4.

42. The method of any one of claims 33-41, wherein the endonuclease is active in a CRISPR/Cas system, wherein the CRISPR/Cas system displays reduced off-target editing activity and maintained on-target editing activity relative to a wild-type CRISPR/Cas system.

43. The modified endonuclease of claim 42, wherein the off-target editing activity is reduced by at least 20%, at least 25%, at least 30%, at least 35%, at least 40%,at least 45%, at least 50%, at least 55%, at least 60%, at least 65%, at least 70%, at least 75%, or at least 80% compared to an unmodified endonuclease.

44. The method of any one of claims 33-43, wherein the mixed charge moiety is covalently linked to a side chain of an amino acid of the endonuclease, to the N-terminal amino group of the endonuclease, and/or to the C-terminal carboxylic group of the endonuclease.

45. The method of any one of claims 33-44, wherein the mixed charge moiety is a peptide with a molecular weight of about 2 kDa to about 130 kDa.

46. The method of claim 45, wherein the modified endonuclease is a fusion protein, wherein the mixed charge moiety is a mixed charge domain consisting of:
a) a plurality of negatively charged amino acids;
b) a plurality of positively charged amino acids; and
c) optionally a plurality of additional amino acids independently selected from the group consisting of proline, serine, threonine, asparagine, glutamine, glycine, and derivatives thereof; and
wherein the ratio of the number of negatively charged amino acids to the number of positively charged amino acids is from about 1:0.5 to about 1:2.

47. The method of claim 46, wherein the plurality of negatively charged amino acids is independently selected from the group consisting of aspartic acid, glutamic acid, and derivatives thereof.

48. The method of claim 46, wherein the plurality of positively charged amino acids is independently selected from the group consisting of lysine, histidine, arginine, and derivatives thereof.

49. The method of claim 46, wherein the mixed charge domain does not comprise a plurality of additional amino acids.

50. The method of claim 46, wherein the plurality of positively charged amino acids are lysines and a plurality of negatively charged amino acids are glutamic acids.

51. The method of claim 46, wherein the mixed charge domain comprises a random sequence.

52. The method of claim 46, wherein the mixed charge domain comprises asequence (X1-X2-X3)ₙ, wherein X1 is a negatively charged amino acid, X2 is a positively charged amino acid, and X3 is absent or is an additional amino acid independently selected from the group consisting of proline, serine, threonine, asparagine, glutamine, glycine, and derivatives thereof, wherein n is an integer from about 5 to about 50.

53. The method of claim 46, wherein the mixed charge domain comprises a plurality of lysines and a plurality of negatively charged amino acids selected from the group consisting of glutamic acid and aspartic acid.

54. The method of claim 46, wherein the mixed charge domain comprises a plurality of histidines and a plurality of negatively charged amino acids selected from the group consisting of glutamic acid and aspartic acid.

55. The method of claim 46, wherein the plurality of additional amino acids is selected from the group consisting of proline, serine, and glycine.

56. The method of claim 46, wherein the plurality of additional amino acids is a plurality of prolines.

57. The method of claim 46, wherein the mixed charge domain comprises a plurality of lysines, a plurality of glutamic acids, and a plurality of prolines.

58. The method of any one of claims 33-44, wherein the mixed charge moiety is a synthetic polymer with a molecular weight of about 2 kDa to about 80 kDa.

59. The method of claim 58, wherein the polymer selected from the group consisting of poly(carboxybetaine) (PCB), poly(sulfobetaine) (PSB), poly(2-methacryloyloxyethyl phosphorylcholine) (PMPC), and poly(tetramethylamine oxide) (TMAO) polymers.

60. The method of claim 58 or claim 59, wherein the polymer is a poly(carboxybetaine) (PCB).


SEQUENCE LISTING (24 sequences)

SEQ ID NO: 1 - DNA, 4101 bp, Artificial sequence
  Synthetic
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 2 - DNA, 4335 bp, Artificial sequence
  Synthetic
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaag

SEQ ID NO: 3 - DNA, 4569 bp, Artificial sequence
  Synthetic
gagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaaggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaag

SEQ ID NO: 4 - DNA, 4803 bp, Artificial sequence
  Synthetic
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaaggagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaaggagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaag

SEQ ID NO: 5 - DNA, 20 bp, Artificial sequence
  Synthetic
gggcacgggcagcttgccgg

SEQ ID NO: 6 - DNA, 20 bp, Artificial sequence
  Synthetic
gggtggggggagtttgctcc

SEQ ID NO: 7 - DNA, 20 bp, Artificial sequence
  Synthetic
ggatggagggagtttgctcc

SEQ ID NO: 8 - DNA, 20 bp, Artificial sequence
  Synthetic
gggagggtggagtttgctcc

SEQ ID NO: 9 - DNA, 20 bp, Artificial sequence
  Synthetic
cgggggagggagtttgctcc

SEQ ID NO: 10 - DNA, 20 bp, Artificial sequence
  Synthetic
ggggaggggaagtttgctcc

SEQ ID NO: 11 - DNA, 20 bp, Artificial sequence
  Synthetic
gagtccgagcagaagaagaa

SEQ ID NO: 12 - DNA, 20 bp, Artificial sequence
  Synthetic
gaggccgagcagaagaaaga

SEQ ID NO: 13 - DNA, 20 bp, Artificial sequence
  Synthetic
gagtcctagcaggagaagaa

SEQ ID NO: 14 - DNA, 20 bp, Artificial sequence
  Synthetic
gagtctaagcagaagaagaa

SEQ ID NO: 15 - DNA, 20 bp, Artificial sequence
  Synthetic
gagttagagcagaagaagaa

SEQ ID NO: 16 - DNA, 20 bp, Artificial sequence
  Synthetic
gcagatgtagtgtttccaca

SEQ ID NO: 17 - DNA, 20 bp, Artificial sequence
  Synthetic
acatatgtagtatttccaca

SEQ ID NO: 18 - DNA, 20 bp, Artificial sequence
  Synthetic
ccagatgtagtattcccaca

SEQ ID NO: 19 - DNA, 20 bp, Artificial sequence
  Synthetic
ctagatgaagtgcttccaca

SEQ ID NO: 20 - DNA, 20 bp, Artificial sequence
  Synthetic
ctagatgaagtgcttccaca

SEQ ID NO: 21 - RNA, 20 nt, Artificial sequence
  Synthetic
aagaauggcuucaagaggcu

SEQ ID NO: 22 - RNA, 20 nt, Artificial sequence
  Synthetic
ucuguaagaauggcuucaag

SEQ ID NO: 23 - RNA, 20 nt, Artificial sequence
  Synthetic
ugguucaucaucuguaagaa

SEQ ID NO: 24 - RNA, 20 nt, Artificial sequence
  Synthetic
acagaugaugaaccagacca